(19) United States
(12) Patent Application Publication
(10) Pub. No.: US 2022/0009822 A1
(43) Pub. Date: Jan. 13, 2022

(54) BIOACTIVE SILICATE GLASSES

(71) Applicant: Corning Incorporated, Corning, NY (US)

(72) Inventor: Qiang Fu, Painted Post, NY (US)

(21) Appl. No.: 17/296,086

(22) PCT Filed: Nov. 18, 2019
(86) PCT No.: PCT/US2019/061988

§ 371 Date: May 21, 2021

Related U.S. Application Data
(60) Provisional application No. 62/771,341, filed on Nov. 26, 2018.
(60) Provisional application No. 62/796,859, filed on Jan. 25, 2019.

(51) Int. Cl. C03C 4/00; C03C 3/097; A61L 26/00; A61L 27/10; A61L 31/02

(52) U.S. Cl. CPC C03C 4/0021; C03C 3/097; C03C 2204/02; A61L 27/10; A61L 31/026; A61L 26/0004

(57) ABSTRACT

An antibacterial composition, includes: a silicate-based glass material having a composition of: 55-70 wt. % SiO₂, 0-10 wt % B₂O₃, 3-18 wt. % P₂O₅, 0-10 wt. % A1₂0₃, 0-5 wt. % Li₂O, 12-30 wt. % Na₂O, 0-15 wt. % K₂O, 0-10 wt. % MgO, 1-15 wt. % CaO, 2-20 wt. % MO, and 15-35 wt. % R₂O, such that MO is the sum of MgO, CaO, SrO, and BaO, such that R₂O is the sum of Na₂O, K₂O, Li₂O, and Rb₂O, and such that the silicate-based glass material can achieve a 6-log kill rate of at least one of E. coli, P. gingivalis, or S. mutans bacteria.

### CROSS-REFERENCE TO RELATED APPLICATIONS

This application claims the benefit of priority of U.S. Provisional Application Ser. No. 62/771,341 filed on Nov. 26, 2018 and U.S. Provisional Application Ser. No. 62/796,859 filed on Jan. 25, 2019 the contents of which are relied upon and incorporated herein by reference in their entirety as if fully set forth below.

### BACKGROUND

### 1. Field

The disclosure relates to bioactive glasses for use in biomedical applications. In particular, the glasses described herein are silicate glasses that show fast filling rates of dentin tubules and have advantageous release rates of metal ions, which provide advantages in antibacterial applications and wound healing.

### 2. Technical Background

Bioactive glasses are a group of glass and glass ceramic materials that have shown biocompatibility or bioactivity, which has allowed them to be incorporated into human or animal physiology. A number of these materials exist on the market already, such as Bioglass 8625, a soda-lime glass used for encapsulation of implanted devices, and Bioglass 45S5, a bioactive glass composition used in bone repair. However, there continues to be an unmet need for solutions to biomedical problems that novel biocompatible inorganic compositions may help resolve.

This disclosure presents improved biocompatible inorganic compositions and methods of manufacturing thereof for biomedical applications.

### SUMMARY

In some embodiments, an antibacterial composition, comprises: a silicate-based glass material having a composition of: 55-70 wt. % SiO₂, 0-10 wt. % B₂O₃, 3-18 wt. % P₂O₅, 0-10 wt. % Al₂O₃, 0-5 wt. % Li₂O, 12-30 wt. % Na₂O, 0-15 wt. % K₂O, 0-10 wt. % MgO, 1-15 wt. % CaO, 2-20 wt. % MO, and 15-35 wt. % R₂O, wherein MO is the sum of MgO, CaO, SrO, and BaO, wherein R₂O is the sum of Na₂O, K₂O, Li₂O, and Rb₂O, and wherein the silicate-based glass material is configured to achieve a 6-log kill rate of at least one of E. coli, P. gingivalis, or S. mutans bacteria.

In one aspect, which is combinable with any of the other aspects or embodiments, the glass material is a particle, bead, particulate, fiber, fibers, woolen mesh, or combination thereof.

In one aspect, which is combinable with any of the other aspects or embodiments, the glass material has at least one size dimension in a range of 1-100 μm.

In one aspect, which is combinable with any of the other aspects or embodiments, the glass material has at least one size dimension in a range of 1-10 μm.

In one aspect, which is combinable with any of the other aspects or embodiments, the glass material has a minimal inhibitory concentration (MIC) of at most 10 mg/mL in the composition.

In one aspect, which is combinable with any of the other aspects or embodiments, the glass material has a minimal inhibitory concentration (MIC) in a range of 10 to 50 mg/mL in the composition.

In one aspect, which is combinable with any of the other aspects or embodiments, the antibacterial composition further comprises: a culture medium having a pH in a range of 7 to 11.

In one aspect, which is combinable with any of the other aspects or embodiments, the antibacterial composition does not comprise silver (Ag)- or copper (Cu)-based materials.

In one aspect, which is combinable with any of the other aspects or embodiments, the antibacterial composition is colorless or essentially colorless.

In one aspect, which is combinable with any of the other aspects or embodiments, the antibacterial composition comprises at least one of: 60-70 wt. % SiO₂, 5-15 wt. % P₂O₅, 15-25 wt. % Na₂O, 0-8 wt. % K₂O, 1-10 wt. % CaO, 0-5 wt. % MgO, or >0-8 wt. % ZnO.

In one aspect, which is combinable with any of the other aspects or embodiments, the antibacterial composition comprises: 59-70 wt. % SiO₂; 5-15 wt. % P₂O₅; and 1-10 wt. % CaO.

In one aspect, which is combinable with any of the other aspects or embodiments, the antibacterial composition comprises: 59-70 wt. % SiO₂; 1-10 wt. % CaO; and is essential free of Al₂O₃.

In one aspect, which is combinable with any of the other aspects or embodiments, the antibacterial composition comprises: 9-70 wt. % SiO₂; 1-10 wt. % CaO; and is essential free of B₂O₃.

In one aspect, which is combinable with any of the other aspects or embodiments, the antibacterial composition further comprises: 0-5 wt. % ZrO₂.

In one aspect, which is combinable with any of the other aspects or embodiments, the antibacterial composition is essentially free of or comprises 1 wt. % or less of Li₂O.

In one aspect, which is combinable with any of the other aspects or embodiments, the antibacterial composition is essentially free of or comprises 1 wt. % or less of B₂O₃, Al₂O₃, and Li₂O.

### BRIEF DESCRIPTION OF THE DRAWINGS

The disclosure will become more fully understood from the following detailed description, taken in conjunction with the accompanying figures, in which:

FIG. 1 illustrates a particle size distribution of Example Composition 5 after air jet milling, according to some embodiments.

FIG. 2 illustrates pH changes of a culture medium as a function of time after soaking with Example Composition 5, according to some embodiments.

FIG. 3 illustrates a bar graph plot of kill rate of E. coli as a function of pH change of a culture medium, according to some embodiments.

FIG. 4 illustrates a bar graph plot of the kill rate of P. Gingivalis after 1, 2 and 7 days of incubation with Example Composition 5, according to some embodiments.

FIG. 5 illustrates a bar graph plot of the kill rate of S. Mutans after 1, 2 and 7 days of incubation with Example Composition 5, according to some embodiments.

### DETAILED DESCRIPTION

In the following description, whenever a group is described as comprising at least one of a group of elements and combinations thereof, it is understood that the group may comprise, consist essentially of, or consist of any number of those elements recited, either individually or in combination with each other. Similarly, whenever a group is described as consisting of at least one of a group of elements or combinations thereof, it is understood that the group may consist of any number of those elements recited, either individually or in combination with each other. Unless otherwise specified, a range of values, when recited, includes both the upper and lower limits of the range as well as any ranges therebetween. As used herein, the indefinite articles “a,” “an,” and the corresponding definite article “the” mean “at least one” or “one or more,” unless otherwise specified. It also is understood that the various features disclosed in the specification and the drawings can be used in any and all combinations.

Where a range of numerical values is recited herein, comprising upper and lower values, unless otherwise stated in specific circumstances, the range is intended to include the endpoints thereof, and all integers and fractions within the range. It is not intended that the scope of the claims be limited to the specific values recited when defining a range. Further, when an amount, concentration, or other value or parameter is given as a range, one or more preferred ranges or a list of upper preferable values and lower preferable values, this is to be understood as specifically disclosing all ranges formed from any pair of any upper range limit or preferred value and any lower range limit or preferred value, regardless of whether such pairs are separately disclosed. Finally, when the term “about” is used in describing a value or an end-point of a range, the disclosure should be understood to include the specific value or end-point referred to. When a numerical value or end-point of a range does not recite “about,” the numerical value or end-point of a range is intended to include two embodiments: one modified by “about,” and one not modified by “about.”

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. It is noted that the terms “substantially” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. Thus, a glass that is “free” or “essentially free” of Al₂O₃ is one in which Al₂O₃ is not actively added or batched into the glass, but may be present in very small amounts as a contaminant (e.g., 500, 400, 300, 200, or 100 parts per million (ppm) or less or).

Herein, glass compositions are expressed in terms of wt % amounts of particular components included therein on an oxide bases unless otherwise indicated. Any component having more than one oxidation state may be present in a glass composition in any oxidation state. However, concentrations of such component are expressed in terms of the oxide in which such component is at its lowest oxidation state unless otherwise indicated.

Unless otherwise specified, all compositions are expressed in terms of weight percent (wt %). Coefficients of thermal expansion (CTE) are expressed in terms of 10⁻⁷/° C., unless otherwise specified. The CTE can be determined, for example, using the procedure described in ASTM E228 “Standard Test Method for Linear Thermal Expansion of Solid Materials with a Push-Rod Dilatometer” or ISO 7991:1987 “Glass—Determination of coefficient of mean linear thermal expansion.” The density in terms of grams/cm³ was measured via the Archimedes method (ASTM C693). Young's modulus, shear modulus, and Poisson's Ratio were measured via the ASTM C623 standard.

Glass Compositions

Bioactive glasses are a group of glass and glass ceramic materials that have shown biocompatibility or bioactivity, which has allowed them to be incorporated into human or animal physiology. The biocompatibility and in vivo properties of the glass are influenced by the glass composition. In the glass compositions described herein, SiO₂ serves as the primary glass-forming oxide in combination with the bioactive oxides of calcium and phosphorous.

In some examples, the glass comprises a combination of SiO₂, Na₂O, P₂O₅, and CaO. In some examples, the glass further comprises Al₂O₃, B₂O₃, K₂O, and/or Li₂O. For example, examples may comprise a glass composition comprising, in wt. %: 55-70 SiO₂, 0-10 B₂O₃, 3-18 P₂O₅, 0-10 Al₂O₃, 0-5 Li₂O, 12-30 Na₂O, 0-15 K_{20, 0}-10 MgO, 1-15 CaO, 2-20 MO, and 15-35 R₂O, wherein MO is the sum of MgO, CaO, SrO, and BaO, R₂O is the sum of Na₂O, K₂O, Li₂O, and Rb₂O. In other examples, the glass composition comprises 60-70 wt. % SiO₂. In some examples, the glass composition comprises 5-15 wt. % P₂O₅. In some examples, the glass composition additionally comprises >0-8 wt. % ZnO. The silicate glasses disclosed herein are particularly suitable for biomedical or bioactive applications.

Silicon dioxide (SiO₂), which is the major oxide component of the embodied glasses, may be included to provide high temperature stability and chemical durability. In some embodiments, the glass can comprise 55-70 wt. % SiO₂. In some examples, the glass may comprise 60-70 wt. % SiO₂. In some examples, the glass can comprise 55-70 wt. %, 55-65 wt. %, 55-60 wt. %, 60-70 wt. %, 60-65 wt. %, or 65-70 wt. % SiO₂. In some examples, the glass is essentially free of SiO₂ or comprises 55, 56, 57, 58, 59, 60, 61, 62, 63, 64, 65, 66, 67, 68, 69, 70 wt. % SiO₂.

In some examples, the glass can comprise 0-10 wt. % B₂O₃. In some examples, the glass can comprise >0-10 wt. % B₂O₃. In some examples, the glass can comprise 0-5 wt. % B₂O₃. In some examples, the glass can comprise from 0-10 wt. %, >0-10 wt. %, 2-10 wt. %, 5-10 wt. %, 0-8 wt. %, >0-8 wt. %, 2-8 wt. %, 5-8 wt. %, 0-5 wt. %, >0-5 wt. %, or 2-5 wt. % B₂O₃. In some examples, the glass can comprise 0, >0, 1, 2, 3, 4, 5, 6, 7, 8, 9, or 10 wt. % B₂O₃.

Alumina (Al₂O₃) may influence the structure of the glass and, additionally, lower the liquidus temperature and coefficient of thermal expansion, or enhance the strain point. In addition to its role as a network former, Al₂O₃ (and ZrO₂) help improve the chemical durability in silicate glass while having no toxicity concerns. In some examples, the glass can comprise 0-10 wt. % Al₂O₃. In some examples, the glass can comprise from 0 to 10 wt. %, 0 to 8 wt. %, 0 to 6 wt. %, 0 to 4 wt. %, 0 to 2 wt. %, >0 to 10 wt. %, >0 to 8 wt. %, >0 to 6 wt. %, >0 to 4 wt. %, >0 to 2 wt. %, 1 to 10 wt. %, 1 to 8 wt. %, 1 to 6 wt. %, 1 to 4 wt. %, 1 to 2 wt. %, 3 to 8 wt. %, 3 to 6 wt. %, 3 to 10 wt. %, 5 to 8 wt. %, 5 to 10 wt. %, 7 to 10 wt. %, or 8 to 10 wt. % Al₂O₃. In some examples, the glass can comprise 0, >0, 1, 2, 3, 4, 5, 6, 7, 8, 9, or 10 wt. % Al₂O₃.

Phosphorus pentoxide (P₂O₅) also serves as a network former. Furthermore, the liberation of phosphate ions to the surface of bioactive glasses contributes to the formation of apatite. The inclusion of phosphate ions in the bioactive glass increases apatite formation rate and the binding capacity of the bone tissue. In addition, P₂O₅ increases the viscosity of the glass, which in turn expands the range of operating temperatures, and is therefore an advantage to the manufacture and formation of the glass. In some examples, the glass can comprise 3-18 wt. % P₂O₅. In some examples, the glass can comprise 3-18 wt. %, 3-15 wt. %, 3-12 wt. %, 3-10 wt. %, 3-8 wt. %, 3-5 wt. %, 5-18 wt. %, 5-15 wt. %, 5-12 wt. %, 5-10 wt. %, 5-8 wt. %, 8-18 wt. %, 8-15 wt. %, 8-12 wt. %, 8-10 wt. %, 10-18 wt. %, 10-15 wt. %, 10-12 wt. %, 12-18 wt. %, or 12-15 wt. % P₂O₅. In some examples, the glass can comprise about 3, 4, 5, 6, 7, 8, 9, 10, 11, 12, 13, 14, 15, 16, 17, or 18 wt. % P₂O₅.

Alkali oxides (Li₂O, Na₂O, K₂O, Rb₂O, or Cs₂O) serve as aids in achieving low melting temperature and low liquidus temperatures. Meanwhile, the addition of alkali oxides can improve bioactivity. Further, Na₂₀ and K₂O may influence the coefficient of thermal expansion, especially at low temperatures. In some examples, the glass can comprise from 12-30 wt. % Na₂O. In some examples, the glass can comprise 15-25 wt. % Na₂O. In some examples, the glass can comprise 20-25 wt. % Na₂₀. In some examples, the glass can comprise from 12-30 wt. %, 12-28 wt. %, 12-25 wt. %, 12-20 wt. %, 12-18 wt. %, 15-30 wt. %, 15-28 wt. %, 15-25 wt. %, 15-20 wt. %, 15-18 wt. %, 18-30 wt. %, 18-28 wt. %, 18-25 wt. %, 18-20 wt. %, 20-30 wt. %, 20-28 wt. %, 20-25 wt. %, or 25-30 wt.° A Na₂O. In some examples, the glass can comprise 12, 13, 14, 15, 16, 17, 18, 19, 20, 21, 22, 23, 24, 25, 26, 27, 28, 29, or 30 wt. % Na₂O.

In some examples, the glass can comprise from 0-15 wt. % K₂O. In some examples, the glass can comprise 0-10 wt. % K₂O. In some examples, the glass can comprise >0-5 wt. % K₂O. In some examples, the glass can comprise from 0-15 wt. %, >0-15 wt. %, 2-15 wt. %, 5-15 wt. %, 8-15 wt. %, 10-15 wt. %, 0-10 wt. %, >0-10 wt. %, 2-10 wt. %, 5-10 wt. %, 0-8 wt. %, >0-8 wt. %, 2-8 wt. %, 5-8 wt. %, 0-5 wt. %, >0-5 wt. %, or 2-5 wt.° A K₂O. In some examples, the glass can comprise 0, >0, 1, 2, 3, 4, 5, 6, 7, 8, 9, 10, 11, 12, 13, 14, or 15 wt. % K₂O.

In some examples, the total amount of Na₂O and K₂₀ is important to the properties of the glass. In such examples, the glass can comprise a total of 15-35 wt. % Na₂O and K₂₀ combined.

In some examples, the glass can comprise from 0-5 wt. % Li₂O. In some examples, the glass can comprise from >0-5 wt. % Li₂O. In some examples, the glass can comprise from about >0-3.5 wt. % Li₂O. In some examples, the glass can comprise from 1-4 wt. % Li₂O. In some examples, the glass can comprise from 0-5 wt. %, 0-4 wt. %, 0-3 wt. %, 0-2 wt. %, >0 to 5 wt. %, >0 to 4 wt. %, >0 to 3 wt. %, >0 to 2 wt. %, 1 to 5 wt. %, 1 to 4 wt. %, or 1 to 3 wt. % Li₂O. In some examples, the glass can comprise about 0, >0, 1, 2, 3, 4, or 5 wt. % Li₂O.

In some examples, the total amount of the alkalis Li₂O, Na₂O, and K₂₀ (R₂O) is important to the glass properties. In some examples, the glass can comprise 12-35 wt. % R₂O, wherein R₂O is the sum or Li₂O, Na₂O, and K₂O. In some examples, the glass can comprise 15-35 wt. % R₂O. In some examples, the glass can comprise from 12-35 wt. %, 12-30 wt. %, 12-28 wt. %, 12-25 wt. %, 12-20 wt. %, 12-18 wt. %, 15-35 wt. %, 15-30 wt. %, 15-28 wt. %, 15-25 wt. %, 15-20 wt. %, 15-18 wt. %, 18-35 wt. %, 18-30 wt. %, 18-28 wt. %, 18-25 wt. %, 20-35 wt. %, 20-30 wt. %, 20-28 wt. %, 20-25 wt. %, 22-35 wt. %, 22-30 wt. %, 22-28 wt. %, 25-35 wt. %, or 25-30 wt. % R₂O. In some examples, the glass can comprise 12, 13, 14, 15, 16, 17, 18, 19, 20, 21, 22, 23, 24, 25, 26, 27, 28, 29, 30, 31, 32, 33, 34, or 35 wt. % R₂O.

Divalent cation oxides (such as alkaline earth oxides) also improve the melting behavior and the bioactivity of the glass. Particularly, CaO is found to be able to react with P₂O₅ to form apatite when immersed in a simulated body fluid (SBF) or in vivo. The release of Ca²⁺ ions from the surface of the glass contributes to the formation of a layer rich in calcium phosphate.

In some examples, the glass can comprise 1-15 wt. % CaO. In some examples, the glass can comprise 1-10 wt. % CaO. In some examples, the glass can comprise 3 to 12 wt. % R₂O. In some examples, the glass can comprise from 1-15 wt. %, 1-12 wt. %, 1-10 wt. %, 1-8 wt. %, 3-15 wt. %, 3-12 wt. %, 3-10 wt. %, 3-8 wt. %, 5-15 wt. %, 5-12 wt. %, 3-10 wt. %, 5-8 wt. %, 8-15 wt. %, 8-12 wt. %, 8-10 wt. %, or 10-15 wt. % Ca0. In some examples, the glass can comprise 1, 2, 3, 4, 5, 6, 7, 8, 9, 10, 11, 12, 13, 14, or 15 wt. % CaO.

The combination of P₂O₅ and CaO may provide advantageous compositions for bioactive glasses. In some examples, the glass compositions comprise P₂O₅ and CaO with the sum of P₂O₅ and CaO being from 6-25 wt. %, 8-20 wt. % or 8-15 wt. %.

In some examples, the glasses comprise MgO. In some examples, the glass can comprise 0-10 wt. % MgO. In some examples, the glass can comprise from 0 to 5 wt. % MgO. In some examples, the glass can comprise from >0 to 10 wt. %, 3 to 10 wt. %, or 3 to 8 wt. % MgO. In some examples, the glass can comprise from 0 to 10 wt. %, 0 to 8 wt. %, 0 to 6 wt. %, 0 to 4 wt. %, 0 to 2 wt. %, >0 to 10 wt. %, >0 to 8 wt. %, >0 to 6 wt. %, >0 to 4 wt. %, >0 to 2 wt. %,1 to 10 wt. %, 1 to 8 wt. %, 1 to 6 wt. %, 1 to 4 wt. %, 1 to 2 wt. %, 3 to 8 wt. %, 3 to 6 wt. %, 3 to 10 wt. %, 5 to 8 wt. %, 5 to 10 wt. %, 7 to 10 wt. %, or 8 to 10 wt. % MgO. In some examples, the glass can comprise about 0, >0, 1, 2, 3, 4, 5, 6, 7, 8, 9, or 10 wt. % MgO.

Strontium oxide (Sr0) may be present in some embodiments and in such examples, the glass can comprise from 0 to 10 wt. % SrO. In some examples, the glass can comprise from >0 to 10 wt. % SrO. In some examples, the glass can comprise from 3 to 10 wt. %, 5 to 10 wt. %, 5 to 8 wt. % SrO. In some examples, the glass can comprise from 0 to 10 wt. %, 0 to 8 wt. %, 0 to 6 wt. %, 0 to 4 wt. %, 0 to 2 wt. %, >0 to 10 wt. %, >0 to 8 wt. %, >0 to 6 wt. %, >0 to 4 wt. %, >0 to 2 wt. %, 1 to 10 wt. %, 1 to 8 wt. %, 1 to 6 wt. %, 1 to 4 wt. %, 1 to 2 wt. %, 3 to 8 wt. %, 3 to 6 wt. %, 3 to 10 wt. %, 5 to 8 wt. %, 5 to 10 wt. %, 7 to 10 wt. %, or 8 to 10 wt. % SrO. In some examples, the glass can comprise about >0, 1, 2, 3, 4, 5, 6, 7, 8, 9, or 10 wt. % Sr0.

Barium oxide (Ba0) may be present in some examples and in such examples, the glass can comprise from 0 to 15 wt. % BaO. In some examples, the glass can comprise from 0 to 10 wt. %, >0 to 5 wt. %, 6 to 13 wt. %, 5 to 15 wt. %, 7 to 13 wt. %, 7 to 11 wt. %, 8 to 12 wt. % BaO. In some examples, the glass can comprise from 0 to 15 wt. %, 0 to 13 wt. %, 0 to 11 wt. %, 0 to 9 wt. %, 0 to 7 wt. %, 0 to 5 wt. %, >0 to 15 wt. %, >0 to 13 wt. %, >0 to 11 wt. %, >0 to 9 wt. %, >0 to 7 wt. %, >0 to 5 wt. %, 1 to 15 wt. %, 1 to 13 wt. %, 1 to 11 wt. %, 1 to 9 wt. %, 1 to 7 wt. %, 1 to 5 wt. %, 3 to 15 wt. %, 3 to 13 wt. %, 3 to 11 wt. %, 3 to 9 wt. %, 3 to 7 wt. %, 3 to 5 wt. %, 5 to 15 wt. %, 5 to 13 wt. %, 5 to 11 wt. %, 5 to 9 wt. %, 5 to 7 wt. %, 7 to 15 wt. %, 7 to 13 wt. %, 7 to 11 wt. %, 7 to 9 wt. %, 9 to 15 wt. %, 9 to 13 wt. %, 9 to 11 wt. %, 11 to 15 wt. %, or 11 to 13 wt. % BaO. In some examples, the glass can comprise about 0, >0, 1, 2, 3, 4, 5, 6, 7, 8, 9, 10, 11, 12, 13, 14, or 15 wt. % BaO.

Alkaline earth oxides may improve other desirable properties in the materials, including influencing the Young's modulus and the coefficient of thermal expansion. In some examples, the glass comprises from 2-20 wt.° A MO (2 wt. % ≤MO≤20 wt. %), where M is the sum of the alkaline earth metals Mg, Ca, Sr, and Ba, in the glass. In some examples, the glass can comprise from 2 to 15 wt. % MO. In some examples, the glass can comprise about 1, 2, 3, 4, 5, 6, 7, 8, 9, 10, 11, 12, 13, 14, 15, 16, 17, 18, 19, or 20 wt. % MO.

In some embodiments, the glasses comprise ZnO. In some examples, the glass can comprise 0-10 wt. % ZnO. In some examples, the glass can comprise from 0 to 5 wt. % ZnO. In some examples, the glass can comprise from >0 to 10 wt. %, 3 to 10 wt. %, or 3 to 8 wt. % ZnO. In some examples, the glass can comprise from 0 to 10 wt. %, 0 to 8 wt. %, 0 to 6 wt. %, 0 to 4 wt. %, 0 to 2 wt. %, >0 to 10 wt. %, >0 to 8 wt. %, >0 to 6 wt. %, >0 to 4 wt. %, >0 to 2 wt. %,1 to 10 wt. %, 1 to 8 wt. %, 1 to 6 wt. %, 1 to 4 wt. %, 1 to 2 wt. %, 3 to 8 wt. %, 3 to 6 wt. %, 3 to 10 wt. %, 5 to 8 wt. %, 5 to 10 wt. %, 7 to 10 wt. %, or 8 to 10 wt. % ZnO. In some examples, the glass can comprise about 0, >0, 1, 2, 3, 4, 5, 6, 7, 8, 9, or 10 wt. % ZnO.

Additional components can be incorporated into the glass to provide additional benefits or may be incorporated as contaminants typically found in commercially-prepared glass. For example, additional components can be added as coloring or fining agents (e.g., to facilitate removal of gaseous inclusions from melted batch materials used to produce the glass) and/or for other purposes. In some examples, the glass may comprise one or more compounds useful as ultraviolet radiation absorbers. In some examples, the glass can comprise 3 wt. % or less ZnO, TiO₂, CeO, MnO, Nb₂O₅, MoO₃, Ta₂O₅, WO₃, SnO₂, Fe₂O₃, As₂O₃, Sb₂O₃, Cl, Br, or combinations thereof In some examples, the glass can comprise from 0 to about 3 wt. %, 0 to about 2 wt. %, 0 to about 1 wt. %, 0 to 0.5 wt. %, 0 to 0.1 wt. %, 0 to 0.05 wt. %, or 0 to 0.01 wt. % ZnO, TiO₂, CeO, MnO, Nb₂O₅, MoO₃, Ta2O₅, WO₃, SnO₂, Fe₂O₃, As₂O₃, Sb₂O₃, Cl, Br, or combinations thereof. The glasses, according to some examples, can also include various contaminants associated with batch materials and/or introduced into the glass by the melting, fining, and/or forming equipment used to produce the glass. For example, in some embodiments, the glass can comprise from 0 to about 3 wt. %, 0 to about 2 wt. %, 0 to about 1 wt. %, 0 to about 0.5 wt. %, 0 to about 0.1 wt. %, 0 to about 0.05 wt. %, or 0 to about 0.01 wt. % SnO₂ or Fe₂O₃, or combinations thereof.

Non-limiting examples of amounts of precursor oxides for forming the embodied glasses are listed in Table 1, along with the properties of the resulting glasses.

TABLE 1

| Oxide (wt %) | 1 | 2 | 3 | 4 | 5 | 6 | 7 |
|---|---|---|---|---|---|---|---|
| SiO₂ | 65.5 | 64.3 | 64.3 | 63.2 | 62.1 | 64.3 | 63.2 |
| B₂O₃ | 0.0 | 0.0 | 0.0 | 0.0 | 0.0 | 0.0 | 0.0 |
| P₂O₅ | 7.4 | 9.1 | 7.3 | 7.1 | 7.0 | 7.3 | 7.1 |
| Al₂O₃ | 0.0 | 0.0 | 0.0 | 0.0 | 0.0 | 0.0 | 0.0 |
| Li₂O | 0.0 | 0.0 | 0.0 | 0.0 | 0.0 | 0.0 | 0.0 |
| Na₂O | 22.5 | 22.1 | 22.1 | 21.7 | 21.3 | 22.1 | 21.7 |
| K₂O | 0.0 | 0.0 | 1.8 | 3.6 | 5.3 | 0.0 | 0.0 |
| MgO | 0.0 | 0.0 | 0.0 | 0.0 | 0.0 | 1.8 | 3.6 |
| CaO | 4.6 | 4.5 | 4.5 | 4.4 | 4.3 | 4.5 | 4.4 |
| ZnO | 0.0 | 0.0 | 0.0 | 0.0 | 0.0 | 0.0 | 0.0 |
| ZrO₂ | 0.0 | 0.0 | 0.0 | 0.0 | 0.0 | 0.0 | 0.0 |
| F⁻ | 0.0 | 0.0 | 0.0 | 0.0 | 0.0 | 0.0 | 0.0 |

The glass compositions disclosed herein can be in any form that is useful for the medical and dental processes disclosed. The compositions can be in the form of, for example, particles, powder, microspheres, fibers, sheets, beads, scaffolds, woven fibers.

Processes for Making Glasses

Glasses having the oxide contents listed in Table 1 can be made via traditional methods. For example, in some examples, the precursor glasses can be formed by thoroughly mixing the requisite batch materials (for example, using a turbular mixer) in order to secure a homogeneous melt, and subsequently placing into silica and/or platinum crucibles. The crucibles can be placed into a furnace and the glass batch melted and maintained at temperatures ranging from 1250-1650° C. for times ranging from about 6-16 hours. The melts can thereafter be poured into steel molds to yield glass slabs. Subsequently, those slabs can be transferred immediately to an annealer operating at about 500-650° C., where the glass is held at temperature for about 1 hour and subsequently cooled overnight. In another non-limiting example, precursor glasses are prepared by dry blending the appropriate oxides and mineral sources for a time sufficient to thoroughly mix the ingredients. The glasses are melted in platinum crucibles at temperatures ranging from about 1100° C. to about 1650° C. and held at temperature for about 16 hours. The resulting glass melts are then poured onto a steel table to cool. The precursor glasses are then annealed at appropriate temperatures.

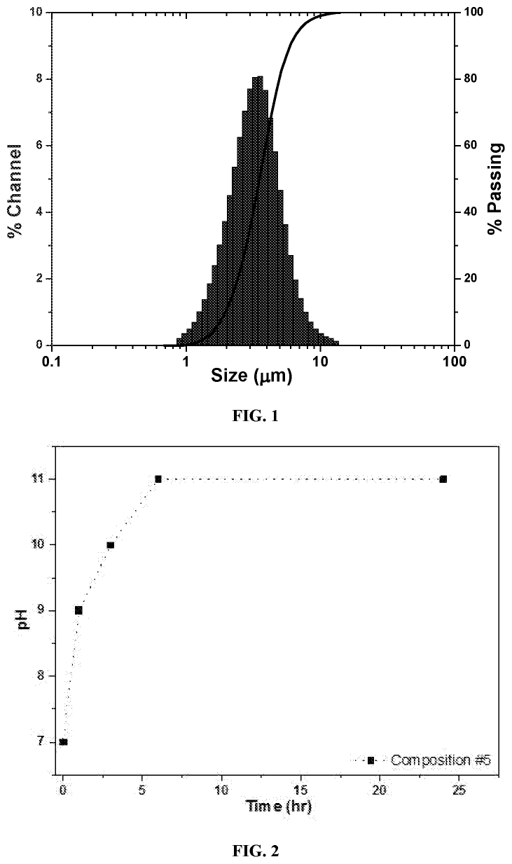

The embodied glass compositions can be ground into fine particles in the range of 1-10 microns (μm) by air jet milling or short fibers. For example, FIG. 1 illustrates a particle size distribution of Example Composition 5 after air jet milling, according to some embodiments. The particle size can be varied in the range of 1-100 _{i—}tm using attrition milling or ball milling of glass frits. Furthermore, these glasses can be processed into short fibers, beads, sheets or three-dimensional scaffolds using different methods. Short fibers are made by melt spinning or electric spinning; beads can be produced by flowing glass particles through a hot vertical furnace or a flame torch; sheets can be manufactured using thin rolling, float or fusion-draw processes; and scaffolds can be produced using rapid prototyping, polymer foam replication and particle sintering. Glasses of desired forms can be used to support cell growth, soft and hard tissue regeneration, stimulation of gene expression or angiogenesis.

Continuous fibers can be easily drawn from the claimed composition using processes known in the art. For example, fibers can be formed using a directly heated (electricity passing directly through) platinum bushing. Glass cullet is loaded into the bushing, heated up until the glass can melt. Temperatures are set to achieve a desired glass viscosity (usually <1000 poise) allowing a drip to form on the orifice in the bushing (Bushing size is selected to create a restriction that influences possible fiber diameter ranges). The drip is pulled by hand to begin forming a fiber. Once a fiber is established it is connected to a rotating pulling/collection drum to continue the pulling process at a consistent speed. Using the drum speed (or revolutions per minute RPM) and glass viscosity the fiber diameter can be manipulated—in general the faster the pull speed, the smaller the fiber diameter. Glass fibers with diameters in the range of 1-100 μm can be drawn continuously from a glass melt. Fibers can also be created using an updraw process. In this process, fibers are pulled from a glass melt surface sitting in a box furnace. By controlling the viscosity of the glass, a quartz rod is used to pull glass from the melt surface to form a fiber. The fiber can be continuously pulled upward to increase the fiber length. The velocity that the rod is pulled up determines the fiber thickness along with the viscosity of the glass.

Glass Bioactivity

Aspects are related to compositions or matrices containing embodied bioactive glass compositions and the methods of using the matrices to treat medical conditions. The matrices can be a toothpaste, mouthwash, rinse, spray, ointment, salve, cream, bandage, polymer film, oral formulation, pill, capsule, transdermal formulation, and the like. The bioactive glass compositions claimed can be physically or chemically attached to matrices or other matrix components, or simply mixed in. As noted above, the bioactive glass can be in any form that works in the application, including particles, beads, particulates, short fibers, long fibers, or woolen meshes. The methods of using the glass-containing matrices to treat a medical condition can be simply like the use of matrix as normally applied.

E. Coli Applications

Glass particles comprising the compositions disclosed herein may demonstrate strong antimicrobial effectiveness against E. coli bacteria. A minimal inhibitory concentration (MIC, corresponding to a 6-log kill rate) as low as 10 mg/mL was determined for Example Composition 5 from Table 1, which is lower than conventional glass particles, such as those with CuNO₃-IOX'd soda lime silicate glass compositions. Table 2 describes the MIC of Example Composition 5 and CuNO₃-IOX'd soda lime silicate glass, both as particles, required to exhibit strong antimicrobial effectiveness against E. coli bacteria.

TABLE 2

| | Example | CuNO₃—IOX'd soda lime |
|---|---|---|
| Particle | Composition 5 | silicate glass |
| MIC (mg/mL) | 10 | 20 |

Testing for the E. coli kill rate was determined by first culturing the test bacteria in a suitable medium for 16 to 18 hrs till a saturation point and calculating bacteria concentration by spectrophotometer. Thereafter, the E. coli bacteria (107 live E. coli) medium is infused with varying concentrations of glass particles. For example, each broth comprises approximately 5 mL of E. coli bacteria medium and 0.5, 1, 10, 20, 50, or 200 mg of glass particles per mL of broth, respectively. In addition, a separate broth comprises E. coli bacteria medium without glass particles. The inoculation of the bacteria/glass is cultured for 24 hrs and thereafter, 10 μL bacteria/broth is plated and cultured for an additional 16 hrs on Agar plates. After culturing, the E. coli colony number for each condition (glass types, concentrations) is counted.

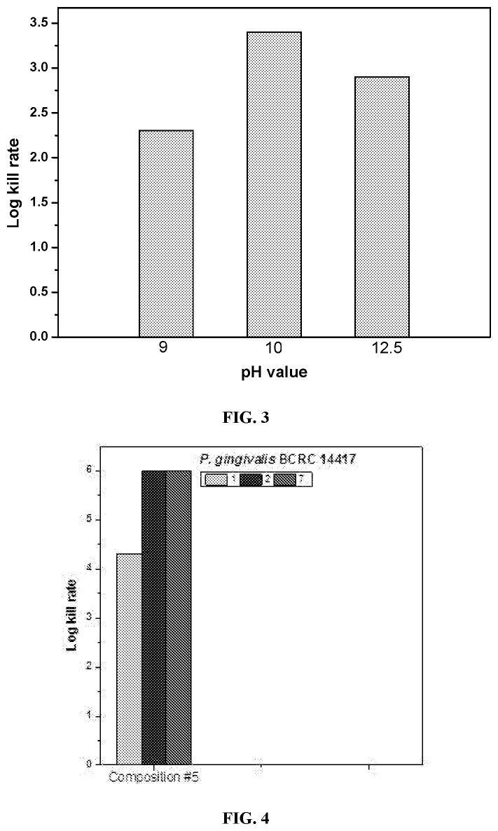

Depending on the Example Glass composition, MIC may vary from 10 to 50 mg/mL. In some examples, antimicrobial effectiveness is associated with alkali and alkali earth ion release from the glass particles. This release may be suggested by pH changes of the broth during testing. For example, FIG. 2 illustrates pH changes of the culture medium as a function of time after soaking with Example Composition 5, according to some embodiments. Rapid pH changes occur from pH=7 to pH=11 within the first 5-7 hrs of culturing and then relatively stabilizes at the upper pH bound thereafter. However, increases of pH alone cannot achieve the required 6-log bacteria kill rates. FIG. 3 illustrates a bar graph plot of kill rate of E. coli as a function of pH change of a culture medium, according to some embodiments. None of pH=9, pH=10, or pH=12.5 culture mediums are able to achieve 6-log bacteria kill rates. Although addition of NaOH to the culture medium shows an inhibitory effect on E. coli growth, a maximum 3.4-log kill rate was reached at pH=10 (FIG. 3). Further increases to pH do not show advantageous inhibitory effect on the bacteria. Without being bound to theory, it is believed that multiple killing mechanisms, which include pH changes and alkali/alkali earth ion-bacterial interaction, account for the excellent antimicrobial effectiveness from the disclosed compositions.

P. Gingivalis/S. Mutans Applications

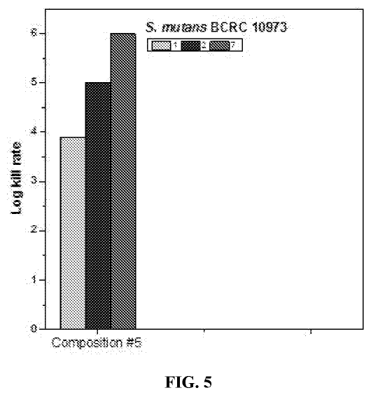

Antibacterial effectiveness may also be demonstrated against other bacteria, such as P. gingivalis and S. mutans, which are two representative oral bacteria in dental applications. P. gingivalis is associated with chronic periodontitis, while S. mutans is associated with oral cavity. All disclosed compositions (for example, those in Table 1) demonstrated a 6-log kill rate after 7 days in medium containing either P. gingivalis or S. mutans bacteria. For example, FIGS. 4 and 5 illustrate bar graph plots of the kill rate of P. Gingivalis and S. Mutans, respectively, after 1, 2 and 7 days of incubation with Example Composition 5, according to some embodiments. Example Composition 5 showed a 4-log kill rate to both bacteria after one day of testing, at least a 5-log kill rate to both bacteria after two days of testing, and a 6-log kill rate to both bacteria after seven days of testing. The antibacterial effectiveness against at least these two representative oral bacteria implies the potential improvement to human oral care using the disclosed glass compositions.

Moreover, because the disclosed compositions (1) show little to no coloring effects of the bacteria-containing medium after soaking for up to 24 hrs or (2) do not contain silver (Ag)- or copper (Cu)-based materials, the compositions are particularly attractive food, water treatment, and paint industry applications.

While typical embodiments have been set forth for the purpose of illustration, the foregoing description should not be deemed to be a limitation on the scope of the disclosure or appended claims. Accordingly, various modifications, adaptations, and alternatives may occur to one skilled in the art without departing from the spirit and scope of the present disclosure or appended claims.

1-16. (canceled)

17. A glass composition comprising, wt %:
55-70 SiO₂
0-10 B2O₃
3-18 P2O₅
0-10 A1₂0₃
0-5 Li₂O
12-30 Na₂O
0-15 K₂O
0-10 MgO
1-15 CaO
2-20 MO, and
15-35 R₂O,
wherein MO is the sum of MgO, CaO, SrO, and BaO, and R₂O is the sum of Na₂O, K₂O, Li₂O, and Rb₂O.

18. The glass composition of claim 17, wherein the glass composition comprises:
60-70 wt % SiO₂.

19. The composition of claim 17, wherein the glass composition comprises:
5-15 wt % P2O₅.

20. The composition of claim 17, wherein the glass composition comprises:
15-25 wt % Na₂O.

21. The glass composition of claim 17, wherein the glass composition comprises:
0-8 wt % K₂O.

22. The glass composition of claim 17, wherein the glass composition comprises:
1-10 wt % CaO.

23. The glass composition of claim 17, wherein the glass composition comprises:
0-5 wt % MgO.

24. The glass composition of claim 17, wherein the glass composition further comprises:
>0-8 wt % ZnO.

25. The glass composition of claim 17, wherein the glass composition comprises:
59-70 wt % SiO_{2;}
5-15 wt % P2O_{5;} and
1-10 wt % CaO.

26. The glass composition of claim 17, wherein the glass composition comprises:
59-70 wt % SiO₂;
1-10 wt % CaO; and
is essential free of Al₂O₃.

27. The glass composition of claim 17, wherein the glass composition comprises:
9-70 wt % SiO₂;
1-10 wt % CaO; and
is essential free of B₂O₃.

28. The glass composition of claim 17, wherein the glass composition further comprises:
0-5 wt % ZrO₂.

29. The glass composition of claim 17, wherein the glass composition is essentially free of or comprises 1 wt % or less of Li₂O.

30. The glass composition of claim 17, wherein the glass composition is essentially free of or comprises 1 wt % or less of B2O₃, Al₂O₃, and Li₂O.

31. The glass composition of claim 17, wherein the glass composition is in the form of powder, particles, beads, particulates, short fibers, long fibers, or woolen meshes.

32-34. (canceled)

35. An oral care composition comprising the glass composition of claim 17.

36. The oral care composition of claim 35, wherein the glass composition is in the form of a plurality of particles, the particles having a particle distribution that is approximately Gaussian and an average particle size of from 10 microns to 100 microns.

37. (canceled)

38. The oral care composition of claim 36, wherein the composition further comprises glycerol, sodium lauryl sulfate, silicon dioxide, polyethylene glycol, and/or a saccharin salt.

39. (canceled)

40. (canceled)

41. A treatment composition for wound care management of a biological tissue, the treatment composition comprising the glass composition of claim 17.

42. The treatment composition of claim 41, wherein the treatment composition is in the form of an article including the composition, for example, a liquid vehicle or solid support, a wound dressing, a stent, an implant, a bandage, an ointment, a salve for oral or topical application, a dosage form for oral or topical administration.

43. (canceled)
